(19) United States
(12) Patent Application Publication
(10) Pub. No.: US 2015/0071884 A1
(43) Pub. Date: Mar. 12, 2015

(54) Nutrition Composition

(71) Applicant: Daniel Lameire, London (GB)

(72) Inventor: Daniel Lameire, London (GB)

(73) Assignee: 33Shake LLP, London (GB)

(21) Appl. No.: 14/482,288

(22) Filed: Sep. 10, 2014

Related U.S. Application Data
(60) Provisional application No. 61/876,001, filed on Sep. 10, 2013.

(51) Int. Cl. A61K 36/9066; A61K 36/55; A61K 36/185; A61K 31/683; A61K 31/047; A61K 36/81; A61K 33/00; A61K 36/05; A61K 36/537; A61K 36/889; A61K 36/54; A61K 36/42; A61K 36/06; A61K 35/74; A61K 36/258; A61K 36/31; A61K 35/64; A61K 36/52; A61K 36/74; A61K 36/48; A23L 1/29

(52) U.S. Cl. CPC A61K 36/9066; A23L 1/296; A61K 36/55; A61K 36/185; A61K 31/683; A61K 31/047; A61K 36/81; A61K 33/00; A61K 36/05; A61K 36/537; A61K 36/889; A61K 36/54; A61K 36/42; A61K 36/06; A61K 35/748; A61K 36/258; A61K 36/31; A61K 35/642; A61K 36/52; A61K 36/74; A61K 36/48
USPC 424/93.4

(57) ABSTRACT

The present invention resides in a composition comprising ingredients providing one or more of: omega 3 and 6 fatty acids; cis fats; vitamins A, B, C, D, E and K; one or more alkalyzing agent; one or more anti-inflammatory agent; one or more immune system enhancing agent; a protein source; one or more antioxidants; one or more blood sugar regulator; one or more sweetener; one or more mineral; and one or more adaptogen. Ideally, such a composition is for use in sport nutrition.

### RELATIONSHIP TO OTHER APPLICATIONS

The present application claims the benefit of and priority to the following application which is hereby incorporated by references for all purposes: U.S. Ser. No. 61/876,001 filed 10 Sep. 2013.

### FIELD OF THE INVENTION

The present invention resides in a composition that is suited to athletes, particularly extreme athletes, to provide complete nutrition after exercise.

The sports nutrition market is dominated by unnatural, manmade, heavily-processed and heavily-sugared products which are also heavy in additives, colorings and preservatives. Even products claiming to be natural rarely are on examination of the list of ingredients.

These products cause many problems for endurance athletes. For example, the core of a real diet for endurance performance must be whole, natural food. The products currently available provide the polar opposite, particularly because the available products are aimed at a mass-market, so cheap ingredients available in bulk are a necessity to keep costs to a minimum.

Sugar adversely affects the body's natural ability to fuel itself efficiently by making the body go to carbohydrates, not stored fat, for fuel. Efficient fueling is the cornerstone of endurance performance and fueling from stored fat as much as possible is the key to this efficiency. So, endurance athletes in particular need to reduce their sugar intake, especially processed sugars and any sugars with a high glycemic index (GI), as much as possible. Due to their high sugar content (all of it high GI), the currently available products do the very opposite.

Endurance athletes need to maintain a good alkaline balance within their bodies. This allows them to fight infections and illness to which they are more vulnerable due to the adverse effects on the immune system of heavy training and racing. If a body is too acidic, the immune system will be weakened. Existing sports nutrition products all make the body more acidic: processed foods, artificial additives and high GI sugars will all acidify the body. As a result, these products actually make the immune problem that endurance athletes face worse.

Artificial, processed and heavily sugared foods are also inflammatory—they increase the amount of inflammation in the body. Endurance events create inflammation through repeated muscular stress over a long period and so such athletes need a diet rich in anti-inflammatory foods to recover properly between racing and training sessions. By the nature of the ingredients of traditional sports nutrition products, inflammation is increased rather than decreased.

Being artificial, manmade and processed, traditional sports nutrition is not healthy. Endurance athletes are health conscious by nature which makes sports nutrition difficult on many levels.

### DESCRIPTION OF THE INVENTION

The present invention seeks to overcome the drawbacks and problems associated with currently available sports nutrition products by providing a composition that provides the athlete with balanced nutrition that is required to support training, exercise and performance.

In particular, the present invention resides in a composition comprising ingredients providing: omega 3 and 6 fatty acids; cis fats; vitamins A, B, C, D, E and K; one or more alkalysing agent; one or more anti-inflammatory agent; one or more immune system enhancing agent; a protein source; one or more antioxidants; one or more blood sugar regulator; one or more sweetener; one or more mineral; and one or more adaptogen.

Optionally, the composition further includes flavoring.

It is particularly preferred that all ingredients are naturally occurring and, as such, substantially all the ingredients are not man-made.

Preferably the composition is suitable for vegetarians, ideally vegans. Thus the protein source is preferably a non-meat protein source.

While the composition of the invention may comprise a specific ingredient for each heading, it will be understood that some natural foods are effective in many different areas. For example, flax seeds provide a source of omega 3, cis fat, vitamins, protein and natural minerals as well as acting as an alkalyzing and anti-inflammatory agent.

Accordingly, the composition of the invention is a natural nutrition product ideally aimed at endurance athletes. The composition has been formulated primarily to meet the nutritional needs of people pushing their bodies to the limit in endurance sports such as marathon and ultramarathon running, triathlon and Ironman racing, and long distance cycling and swimming events. However, it will be appreciated that under-nourished or sick humans may also derive equal benefit from the composition.

Through its specific combination of ingredients, the composition of the present invention provides the following benefits:
1. alkalyze the body and boost the immune system to combat immune issues associated with heavy training and racing;
2. increase the body's ability to burn fat for increased efficiency; and
3. provide the body with natural anti-inflammatories for faster recovery and better performance; and

In a particularly preferred embodiment, the components of the composition are substantially all derived from a natural source, making the composition genuinely healthy in addition to its specific endurance properties.

Suitable sources of omega 3 and 6 fatty acids include flax seeds, hemp seeds, lecithin, walnuts, sesame seeds, Chia seeds, pumpkin seeds, and Spirulina.

Cis fats are naturally occurring unsaturated fats that are generally considered to be “good” fats. Indeed, unsaturated (cis) fats increase the levels of good cholesterol (high density lipoprotein (HDL)) in the body. Suitable sources of cis fats for the purpose of present invention include flax seeds, hemp seeds, coconut, walnuts, sesame seeds and pumpkin seeds.

Suitable sources of vitamins A, B, C, D, E and K include flaxseeds, hemp seeds, coconut, Goji berries, walnuts, bee pollen, sesame seeds, cranberries, maca, cinnamon, pumpkin seeds, blueberries, broccoli, nutritional yeast, Spirulina, pomegranate, turmeric, Acai berries, Chlorella, beetroot, Chia seeds and wheatgrass.

At least one alkalyzing agent is necessary for the composition to balance acid generated through exercise. Suitable alkalyzing agents include flaxseeds, hemp seeds, lecithin, birch tree sugar (Xylitol), sesame seeds, Incan berries, baking soda, barleygrass, Chlorella and Chia seeds.

Anti-inflammatory agents may be found in natural foods such as flaxseeds, hemp seeds, coconut, cinnamon, pumpkin seeds, nutritional yeast, Spirulina, pomegranate, turmeric, ginseng, Chia seeds and broccoli.

Foods that support, enhance or boost the immune system naturally include ginseng, cacao, coconut, bee pollen, cinnamon, Reishi mushrooms and walnuts.

While any protein source may be provided, it is particularly preferred if the protein is from a non-meat source, such as flax seeds, hemp seeds, nutritional yeast, Spirulina, Acai berries, Chlorella, Chia seeds, Goji berries, walnuts, sesame seeds, maca, pumpkin seeds, Incan berries, carob, bee pollen, Reishi mushrooms and/or wheatgrass.

Suitable antioxidants include Acai berries, green coffee beans, cacao, carob, Chia seeds, spelt, cranberries, maca, cinnamon, Incan berries, blueberries, broccoli, Spirulina, pomegranate, turmeric, ginseng, Chlorella and beetroot.

Suitable regulators of blood sugar include green coffee beans, carob, coconut, birch tree sugar and Chia seeds.

Natural sweeteners may be found in coconut, Goji berries, birch tree sugar (Xylitol), coconut palm sugar, cranberries, Incan berries, pomegranate, Acai berries, vanilla, cacao and carob.

Minerals that are particularly important include one or more of calcium, phosphorous, magnesium, manganese and/or zinc. It is particularly preferred if the minerals in the composition are derived from natural sources, for example, spelt, sesame seeds, pumpkin seeds, Spirulina, turmeric, Acai berries, Himalayan pink salt, cacao, flax seeds, hemp seeds, coconut, bee pollen, cranberries, maca, broccoli, Chia seeds and/or wheatgrass.

Adaptogens help the body deal with new demands and increased stresses. Suitable adaptogens are ideally provided naturally in sources including Goji berries, maca, Incan berries, ginseng and/or Chlorella.

While the ingredients listed above all provide suitable and palatable flavor, additional flavor may be added, for example by the addition of vanilla, cranberries, blueberries, cacao, carob and coffee, such as roasted coffee beans, as ingredients or as flavoring.

The ingredients ideally incorporated into the composition in their natural form, but may also be in dried (or desiccated) form such as freeze dried, air dried or lyophilized. If in dried form, the ingredients may be whole, diced, macerated, pulped or powdered.

It is particularly preferred that the ingredients are in their raw format and are subjected to minimal processing to maximize the nutritional benefit.

It is preferred if as many of the ingredients as possible are organic.

The composition may be supplied in any suitable form, such as pre-mixed in liquid form or as a syrup, concentrate, essence, powder or crystals that can be made up into a liquid by the consumer, or as a dry, wet or semi-moisturized mixture of raw ingredients.

A particularly preferred composition comprises coconut, hemp seeds, spelt, lecithin, flax seeds, birch tree sugar, coconut palm sugar, Goji berries, walnuts, cranberries, sesame seeds, pumpkin seeds, Incan berries, bee pollen, maca, cinnamon, nutritional yeast, blueberries, xanthum gum, broccoli, baking soda, turmeric, Acai berries, Reishi mushrooms, barley grass, green coffee beans, ginseng, Spirulina, chlorella, beetroot, vanilla, pink Himalayan salt and pomegranate.

Such composition comprises the above list of ingredients in the following percentages: coconut 5-20% preferably 11%, hemp seeds 5-20% preferably 12.5%, spelt 5-15% preferably 9%, lecithin 5-20% preferably 11%, flax seeds 5-10% preferably 7%, birch tree sugar (Xylitol) 5-15% preferably 9%, coconut palm sugar 5-15% preferably 9%, Goji berries 1-8% preferably 4%, walnuts 1-8% preferably 4%, cranberries 1-8% preferably 4%, sesame seeds 0.5-5% preferably 1%, pumpkin seeds 0.5-5% preferably 1%, Incan berries 0.5-5% preferably 1%, bee pollen 0.5-5% preferably 1%, maca 0.5-5% preferably 1%, cinnamon 0.5-5% preferably 1%, nutritional yeast 0.5-5% preferably 1%, blueberries 0.5-5% preferably 1%, xanthum gum 0.5-5% preferably 1%, broccoli 0.5-5% preferably 1%, baking soda 0.5-5% preferably 1%, turmeric 0.5-5% preferably 1%, Acai berries 0.5-5% preferably 1%, Reishi mushroom 0.5-5% preferably 1%, barley grass 0.5-5% preferably 1%, green coffee beans 0.5-5% preferably 1%, ginseng 0.5-5% preferably 1%, Spirulina 0.5-5% preferably 1%, chlorella 0.5-5% preferably 1%, beetroot 0.5-5% preferably 1%, vanilla 0.5-5% preferably 1%, pink Himalayan salt 0.1-2% preferably 0.5%, and pomegranate 0.5-5% preferably 1%.

An alternative composition comprises coconut, hemp seeds, spelt, lecithin, flax seeds, birch tree sugar, coconut palm sugar, Goji berries, walnuts, cranberries, sesame seeds, pumpkin seeds, Incan berries, maca, cinnamon, nutritional yeast, blueberries, xanthum gum, baking soda, turmeric, Acai berries, Reishi mushrooms, barley grass, green coffee beans, ginseng, Spirulina, chlorella, beetroot, vanilla, pink Himalayan salt, pomegranate cacao and carob.

Such composition comprises the above list of ingredients in the following percentages: coconut 5-20% preferably 11%, hemp seeds 5-20% preferably 12.5%, spelt 5-15% preferably 9%, lecithin 5-20% preferably 11%, flax seeds 5-10% preferably 7%, birch tree sugar (Xylitol) 5-15% preferably 9%, coconut palm sugar 5-15% preferably 9%, Goji berries 1-8% preferably 4%, walnuts 1-8% preferably 4%, cranberries 1-8% preferably 4%, sesame seeds 0.5-5% preferably 1%, pumpkin seeds 0.5-5% preferably 1%, Incan berries 0.5-5% preferably 1%, maca 0.5-5% preferably 1%, cinnamon 0.5-5% preferably 1%, nutritional yeast 0.5-5% preferably 1%, blueberries 0.5-5% preferably 1%, xanthum gum 0.5-5% preferably 1%, baking soda 0.5-5% preferably 1%, turmeric 0.5-5% preferably 1%, Acai berries 0.5-5% preferably 1%, Reishi mushroom 0.5-5% preferably 1%, barley grass 0.5-5% preferably 1%, green coffee beans 0.5-5% preferably 1%, ginseng 0.5-5% preferably 1%, Spirulina 0.5-5% preferably 1%, Chlorella 0.5-5% preferably 1%, beetroot 0.5-5% preferably 1%, vanilla 0.5-5% preferably 1%, pink Himalayan salt 0.1-2% preferably 0.5%, pomegranate 0.5-5% preferably 1%, cacao 10-20% preferably 14%, and carob 1-10% preferably 5%.

An alternative composition comprises coconut, hemp seeds, spelt, lecithin, flaxseeds, birch tree sugar, coconut palm sugar, Goji berries, walnuts, cranberries, sesame seeds, pumpkin seeds, Incan berries, maca, cinnamon, nutritional yeast, blueberries, xanthum gum, baking soda, turmeric, Acai berries, Reishi mushrooms, barleygrass, green coffee beans, ginseng, Spirulina, Chlorella, beetroot, vanilla, pink Himalayan salt, pomegranate cacao and roasted coffee beans.

Such composition comprises the above list of ingredients in the following percentages: coconut 5-20% preferably 11%, hemp seeds 5-20% preferably 12.5%, spelt 5-15% preferably 9%, lecithin 5-20% preferably 11%, flax seeds 5-10% preferably 7%, birch tree sugar (Xylitol) 5-15% preferably 9%, coconut palm sugar 5-15% preferably 9%, Goji berries 1-8% preferably 4%, walnuts 1-8% preferably 4%, cranberries 1-8% preferably 4%, sesame seeds 0.5-5% preferably 1%, pumpkin seeds 0.5-5% preferably 1%, Incan berries 0.5-5% preferably 1%, maca 0.5-5% preferably 1%, cinnamon 0.5-5% preferably 1%, nutritional yeast 0.5-5% preferably 1%, blueberries 0.5-5% preferably 1%, xanthum gum 0.5-5% preferably 1%, baking soda 0.5-5% preferably 1%, turmeric 0.5-5% preferably 1%, Acai berries 0.5-5% preferably 1%, Reishi mushroom 0.5-5% preferably 1%, barleygrass 0.5-5% preferably 1%, green coffee beans 0.5-5% preferably 1%, ginseng 0.5-5% preferably 1%, Spirulina 0.5-5% preferably 1%, chlorella 0.5-5% preferably 1%, beetroot 0.5-5% preferably 1%, vanilla 0.5-5% preferably 1%, pink Himalayan salt 0.1-2% preferably 0.5%, pomegranate 0.5-5% preferably 1%, cacao 10-20% preferably 14%, and roasted coffee beans 0.5-5% preferably 2%.

The specific compositions listed above may further or alternatively include Chia seeds, ideally in an amount of between about 8 and 20%, preferably 12%, and/or wheatgrass, ideally in an amount of between about 0.5 and 5%, preferably 2%.

The specific ingredients listed above have been selected for their efficient properties.

Flax seeds are good fat cholesterol busters. They are massively anti-inflammatory for excellent post-training or racing recovery, as well as being full of good fats (including 400% of your daily heart-healthy alpha-linolenic acid among others). Flax seeds are one of the highest known vegetable sources of omega 3, as well as having the ability to lower cholesterol.

Hemp seeds are a complete food on their own, being roughly one third essential fatty acids (omega 3 and 6), one third heart healthy fiber, and one third protein (containing all twenty amino acids), as well as many vitamins.

Spelt are described as being the perfect wholegrain, providing wholegrain goodness for lower cholesterol and heart benefits, essential minerals and good antioxidant properties.

Coconut has more good fat than you can shake a stick at. Packed with medium-chain triglycerides for metabolism-boosting fat loss, raw coconut is also rich in dietary fiber to prevent blood sugar swings and lower blood pressure and relieves stress. It also boosts the immune system and is loaded with essential minerals.

Lecithin is a cholesterol-buster. Not only does lecithin pull down cholesterol levels and break down fat, but it is also loaded with omega 3 and 6 fatty acids and boosts cardiovascular function.

Goji berries are the world's most powerful berries with more vitamin C than oranges, more beta-carotene than carrots, as well as eighteen amino acids including all eight essential amino acids. In addition, they are powerful adaptogens, promoting immune system function and athletic performance.

Walnuts are the King of Nuts, providing a massive source of great omega 3 fatty acids, plus vitamin E for protection against free radicals produced by heavy training, a stack of B vitamins to stoke metabolism and magnesium to regulate blood pressure

Birch tree sugar (Xylitol) is the best sugar-substitute in the world. Extracted from birch tree bark, this sweetener prevents cavities, stabilizes insulin levels, is slowly absorbed into the body and stabilizes hormone levels.

Coconut palm sugar is a low-GI, high-nutrition sweetener.

Bee pollen provides sources of protein, carbohydrates and vitamins A, C and D, as well as all the B vitamins except B12. It is an endurance energy booster that wards off hunger pangs.

Sesame seeds are great for your heart, full of calcium, phosphorous and magnesium to promote bone strength, and provide a great natural protein source.

Cranberries act as a powerful antioxidant, providing vitamin C, fiber, manganese and vitamin K.

Lepidium meyenii, commonly known as maca, is a herbaceous biennial plant of the crucifer family native to the high Andes of Peru around Lake Junin. It is grown for its fleshy hypocotyl (a fused hypocotyl and taproot), which is used as a root vegetable, a medicinal herb, and a supposed aphrodisiac. Low in fat, high in protein while also packing vitamin C and calcium, maca is a physical and mental energizer that is adaptogenic too, meaning it helps the body adapt naturally to changing physical demands.

Cinnamon has been valued for centuries for its medical properties strengthening the immune system, regulating blood consistency, and acting as a major anti-inflammatory agent.

Pumpkin seeds are low fat, low sugar, high protein (25%), loads of essential minerals, including iron and zinc, and a stash of B vitamins. They also have anti-inflammatory properties (strong), cholesterol-reducing abilities (powerful) and mood-enhancing strengths (happy).

Incan berries are crammed with fiber (almost twice as much as dates) and good carbohydrates, as well as a decent hit of protein and vitamin C, helping alkalyze the body and maintain cell function.

Blueberries are ranked as having one of the highest antioxidant capacities among all fruits, vegetables, spices and seasonings. Antioxidants are essential to optimizing health by helping to combat the free radicals that can damage cellular structures as well as DNA. Blueberries also have a low GI index and so are good at regulating blood sugar levels.

Broccoli is considered to be one of the most nutritious vegetables that can offer numerous health benefits. Broccoli contains a substantial amount of nutrients that are extremely good for health including vitamins A, C, folic acid, fiber and calcium. This cruciferous vegetable has also become popular because of its ability to offer the following health benefits. The high potassium content in broccoli is believed to maintain a healthier nervous system as well as allowing the human brain to function optimally. It also works to promote the regular growth of muscles. In addition, broccoli provides high levels of magnesium, calcium and potassium, which are good for regulating blood pressure, as well as a huge amount of soluble fiber which is proven to be effective in drawing cholesterol from the body.

Made from molasses, nutritional yeast is an excellent protein source is and full of B complex vitamins, which also beats stress.

Being 60% protein, Spirulina makes steak (just 22% protein) look pretty useless. Add high levels of vitamin B12, vitamin E, vitamin A (15 times more than carrots), calcium and omega 3 and 6 and you've got the best single food source on the planet.

Pomegranates are full of antioxidants. These are compounds and enzymes known for keeping low-density lipoprotein (LDL) or “bad” cholesterol from oxidizing and causing atherosclerosis, or hardening of the arteries. Pomegranate seeds act a lot like aspirin, keeping blood platelets from sticking together and forming dangerous blood clots. Research has also shown that eating organic pomegranate seeds and drinking pomegranate juice can increase oxygen levels to the heart.

Baking soda not only alkalizes the body, it also reduces the acid impact of acidic foods so common in the modern diet.

A great detoxifier, turmeric is full of essential nutrients including calcium, iron and potassium. It is also a major anti-inflammatory and antioxidant which is why it has long been a trusted holistic healer.

Like blueberries, Acai berries have serious anti-oxidizing properties and are also packed with potassium which aids muscle growth, busts stress and helps optimize metabolism to extract the best from everything you eat.

In Chinese, Reishi mushroom means “mushroom of immortality”, while in Japan the government recognizes it officially as a treatment for cancer. Reishi mushrooms are rich in immune system stimulating polysaccharides, triterpenoids and specialized proteins that have stimulating effects on immune system function

Barleygrass is an alkaline superstar, like wheatgrass but better. Packed with energy this boosts alkalinity, detoxifies and helps slow down ageing.

Green coffee beans are the raw version of coffee and are how coffee starts life. They assist in blood glucose management, reduce blood sugar levels, promote good sleep and protect against heart disease.

Ginseng helps athletes use oxygen more effectively and is believed to regulate metabolism, which can increase energy levels. Consumption of ginseng can also help athletes lower their recovery time and reduce stress. In addition, ginseng can act as a stimulant for the brain cells and help improve concentration and thinking ability. Ginseng also increases a sense of wellbeing and stamina, improves both mental and physical performance and reduces mental and physical stress. It is often used for lowering blood glucose levels and controlling blood pressure. Because of its adaptogenic effects, ginseng is widely used to lower cholesterol, increase energy and endurance, reduce fatigue and the effects of stress, and prevent infections. Ginseng is one of the most effective anti-aging supplements, with the capability of alleviating some major effects of aging such as degeneration of the blood system, and increasing mental and physical capacity.

With over twice as much protein as chicken and stacked full of all the goodness of the best green veggies, Chlorella is a powerful alkalyzer, as well as a detoxifying, fortifying gem. Chlorella is a single celled green alga that is an attractive potential food source because it is high in protein and other essential nutrients. When dried, it is about 45% protein, 20% fat, 20% carbohydrate, 5% fiber, and 10% minerals and vitamins.

Not only is beetroot great for boosting stamina and making muscles work harder, it also contains potassium, magnesium and iron as well as vitamins A, B6 and C, and folic acid. Beetroot also contains carbohydrates, protein, powerful antioxidants and soluble fiber.

Vanilla is a natural, tasty, sweetener with added carbohydrate and vitamins.

Xanthum gum is a gluten-free texture-maker. A natural carbohydrate made from a microorganism called Xanthomonas Campetris, xanthum gum is the perfect gluten-free solution for thickening.

Pink Himalayan salt is the purest salt there is. With no additives and containing the eighty-four naturally-occurring minerals needed for survival, this is salt as pure and natural as it should be.

Raw organic cacao is an antioxidant powerhouse. One of the most antioxidant-rich foods on earth, raw organic cacao is a superfood superstar. Rich in minerals, it also fights free radicals and stimulates endorphin release.

As well as being high in anti-oxidants, carob is rich in phosphorus and calcium, and contains Vitamin E. It is effective in lowering cholesterol level in the blood and works as an analgesic, anti-allergic, antibacterial, antioxidant, antiviral and antiseptic. Carob also has stabilizing and thickening properties and is widely used as a low-fat alternative to cocoa powder as flavoring.

Roasted coffee beans have powerful anti-oxidant properties as well as providing flavoring.

Chia seeds (from Salvia hispanica) are high in fiber and omega 3 for stamina and endurance. They also come with a big hit of antioxidants and are anti-inflammatory and a mood-booster.

Wheatgrass contains over ninety minerals, including high concentrations of the most alkaline minerals, potassium, calcium, magnesium and sodium. It contains the essential enzymes protease (assists in protein digestion), cytochrome oxidase (a powerful anti oxidant), amylase (facilitates digestion), lipase (a fat splitting enzyme), transhydrogenase (strengthens the heart muscle) and superoxide dismutase (found in all body cells and is known for its ability to slow cellular aging). Wheatgrass has more vitamin C than oranges and twice the vitamin A as carrots. It helps the body build red blood cells, increasing endurance during physical exercise, and contains nineteen amino acids, providing the building blocks of protein.

It will be appreciated that the specific ingredients listed above may be substituted for ingredients that have the same effect and/or provide the same nutritional value.

1. A composition comprising ingredients providing one or more of: omega 3 and 6 fatty acids; cis fats; vitamins A, B, C, D, E and K; one or more alkalyzing agent; one or more anti-inflammatory agent; one or more immune system enhancing agent; a protein source; one or more antioxidants; one or more blood sugar regulator; one or more sweetener; one or more mineral; and one or more adaptogen.

2. The composition of claim 1, wherein omega 3 and 6 fatty acids are provided by one or more of flax seeds, hemp seeds, lecithin, walnuts, sesame seeds, Chia seeds, pumpkin seeds, and/or Spirulina.

3. The composition of claim 1, wherein cis fats are provided by one or more of flaxseeds, hemp seeds, coconut, walnuts, sesame seeds and/or pumpkin seeds.

4. The composition of claim 1, wherein vitamins A, B, C, D, E and K are provided by one or more of flax seeds, hemp seeds, coconut, Goji berries, walnuts, bee pollen, sesame seeds, cranberries, maca, cinnamon, pumpkin seeds, blueberries, broccoli, nutritional yeast, Spirulina, pomegranate, turmeric, Acai berries, Chlorella, beetroot, Chia seeds and/or wheatgrass.

5. The composition of claim 1, wherein the one or more alkalyzing agent is provided by flaxseeds, hemp seeds, lecithin, birch tree sugar (Xylitol), sesame seeds, Incan berries, baking soda, barleygrass, Chlorella and/or Chia seeds.

6. The composition of claim 1, wherein the one or more anti-inflammatory agent is provided by one or more of flax seeds, hemp seeds, coconut, cinnamon, pumpkin seeds, nutritional yeast, Spirulina, pomegranate, turmeric, ginseng, Chia seeds and/or broccoli.

7. The composition of claim 1, wherein the one or more immune system enhancing agent is provided by one or more of ginseng, cacao, coconut, bee pollen, cinnamon, Reishi mushrooms and walnuts.

8. The composition of claim 1, wherein the protein source is from a non-meat source, preferably provided by one or more of flaxseeds, hemp seeds, nutritional yeast, Spirulina, acai berries, Chlorella, Chia seeds, Goji berries, walnuts, sesame seeds, maca, pumpkin seeds, Incan berries, carob, bee pollen, Reishi mushrooms and/or wheatgrass.

9. The composition of claim 1, wherein the one or more antioxidant is provided by one or more of Acai berries, green coffee beans, cacao, carob, Chia seeds, spelt, cranberries, maca, cinnamon, Incan berries, blueberries, broccoli, Spirulina, pomegranate, turmeric, ginseng, Chlorella and/or beetroot.

10. The composition of claim 1, wherein the one or more regulator of blood sugar is provided by one or more of green coffee beans, carob, coconut, birch tree sugar and/or Chia seeds.

11. The composition of claim 1, wherein the one or more sweetener is provided by one or more of coconut, Goji berries, birch tree sugar (Xylitol), coconut palm sugar, cranberries, Incan berries, pomegranate, Acai berries, vanilla, cacao and/or carob.

12. The composition of claim 1, wherein the one or more mineral is calcium, phosphorous, magnesium, manganese and/or zinc.

13. The composition of claim 1, wherein the one or more mineral is provided by one or more of spelt, sesame seeds, pumpkin seeds, Spirulina, turmeric, Acai berries, Himalayan pink salt, cacao, flax seeds, hemp seeds, coconut, bee pollen, cranberries, maca, broccoli, Chia seeds and/or wheatgrass.

14. The composition of claim 1, wherein the one or more adaptogen is provided by Goji berries, maca, Incan berries, ginseng and/or Chlorella.

15. The composition of claim 1, wherein the flavoring is provided by vanilla, cranberries, blueberries, cacao, carob and/or roasted coffee beans.

16. The composition of claim 1 comprising coconut preferably 5-20%, more preferably 11% of the total composition, hemp seeds preferably 5-20%, more preferably 12.5% of the total composition, spelt preferably 5-15%, more preferably 9% of the total composition, lecithin preferably 5-20%, more preferably 11% of the total composition, flax seeds preferably 5-10%, more preferably 7% of the total composition, birch tree sugar (Xylitol) preferably 5-15%, more preferably 9% of the total composition, coconut palm sugar preferably 5-15%, more preferably 9% of the total composition, Goji berries preferably 1-8%, more preferably 4% of the total composition, walnuts preferably 1-8%, more preferably 4% of the total composition, cranberries preferably 1-8%, more preferably 4% of the total composition, sesame seeds preferably 0.5-5%, more preferably 1% of the total composition, pumpkin seeds preferably 0.5-5%, more preferably 1% of the total composition, Incan berries preferably 0.5-5%, more preferably 1% of the total composition, bee pollen preferably 0.5-5%, more preferably 1% of the total composition, maca preferably 0.5-5%, more preferably 1% of the total composition, cinnamon preferably 0.5-5%, more preferably 1% of the total composition, nutritional yeast preferably 0.5-5%, more preferably 1% of the total composition, blueberries preferably 0.5-5%, more preferably 1% of the total composition, xanthum gum preferably 0.5-5%, more preferably 1% of the total composition, broccoli preferably 0.5-5%, more preferably 1% of the total composition, baking soda preferably 0.5-5%, more preferably 1% of the total composition, turmeric preferably 0.5-5%, more preferably 1% of the total composition, Acai berries preferably 0.5-5%, more preferably 1% of the total composition, Reishi mushrooms preferably 0.5-5%, more preferably 1% of the total composition, barley grass preferably 0.5-5%, more preferably 1% of the total composition, green coffee beans preferably 0.5-5%, more preferably 1% of the total composition, ginseng preferably 0.5-5%, more preferably 1% of the total composition, Spirulina preferably 0.5-5%, more preferably 1% of the total composition, Chlorella preferably 0.5-5%, more preferably 1% of the total composition, beetroot preferably 0.5-5%, more preferably 1% of the total composition, vanilla preferably 0.5-5%, more preferably 1% of the total composition, pink Himalayan salt preferably 0.1-2%, more preferably 0.5% of the total composition and pomegranate preferably 0.5-5%, more preferably 1% of the total composition.

17. The composition of claim 1 comprising coconut preferably 5-20%, more preferably 11% of the total composition, hemp seeds preferably 5-20%, more preferably 12.5% of the total composition, spelt preferably 5-15%, more preferably 9% of the total composition, lecithin preferably 5-20%, more preferably 11% of the total composition, flax seeds preferably 5-10%, more preferably 7% of the total composition, birch tree sugar (Xylitol) preferably 5-15%, more preferably 9% of the total composition, coconut palm sugar preferably 5-15%, more preferably 9% of the total composition, Goji berries preferably 1-8%, more preferably 4% of the total composition, walnuts preferably 1-8%, more preferably 4% of the total composition, cranberries preferably 1-8%, more preferably 4% of the total composition, sesame seeds preferably 0.5-5%, more preferably 1% of the total composition, pumpkin seeds preferably 0.5-5%, more preferably 1% of the total composition, Incan berries preferably 0.5-5%, more preferably 1% of the total composition, maca preferably 0.5-5%, more preferably 1% of the total composition, cinnamon preferably 0.5-5%, more preferably 1% of the total composition, nutritional yeast preferably 0.5-5%, more preferably 1% of the total composition, blueberries preferably 0.5-5%, more preferably 1% of the total composition, xanthum gum preferably 0.5-5%, more preferably 1% of the total composition, baking soda preferably 0.5-5%, more preferably 1% of the total composition, turmeric preferably 0.5-5%, more preferably 1% of the total composition, Acai berries preferably 0.5-5%, more preferably 1% of the total composition, Reishi mushrooms preferably 0.5-5%, more preferably 1% of the total composition, barleygrass preferably 0.5-5%, more preferably 1% of the total composition, green coffee beans preferably 0.5-5%, more preferably 1% of the total composition, ginseng preferably 0.5-5%, more preferably 1% of the total composition, Spirulina preferably 0.5-5%, more preferably 1% of the total composition, Chlorella preferably 0.5-5%, more preferably 1% of the total composition, beetroot preferably 0.5-5%, more preferably 1% of the total composition, vanilla preferably 0.5-5%, more preferably 1% of the total composition, pink Himalayan salt preferably 0.1-2%, more preferably 0.5% of the total composition, pomegranate preferably 0.5-5%, more preferably 1% of the total composition, cacao preferably 10-20%, more preferably 14% of the total composition, carob preferably 1-10%, more preferably 5% of the total composition and optionally roasted coffee beans preferably 0.5-5%, more preferably 2% of the total composition.

18. The composition of claim 16, the composition further or alternatively including Chia seeds preferably provided in an amount of between about 8 and 20%, more preferably 12% of the total composition and/or wheatgrass preferably provided in an amount of between about 0.5 and 5%, more preferably 2% of the total composition.

19. The composition of claim 17, the composition further or alternatively including Chia seeds preferably provided in an amount of between about 8 and 20%, more preferably 12% of the total composition and/or wheatgrass preferably provided in an amount of between about 0.5 and 5%, more preferably 2% of the total composition.

20. The composition of claim 1, wherein ingredients are pre-mixed in liquid form or are provided as a syrup, concentrate, essence, powder or crystals.
